(19) United States
(12) Patent Application Publication
(10) Pub. No.: US 2010/0291200 A1
(43) Pub. Date: Nov. 18, 2010

(54) FORMULATIONS FOR POORLY SOLUBLE DRUGS

(75) Inventors: Shlomo MAGDASSI, Jerusalem (IL); Yoram SELA, Raanana (IL); Keren COHEN, Jerusalem (IL)

Correspondence Address: THE NATH LAW GROUP, 112 South West Street, Alexandria, VA, 22314, US

(73) Assignee: LYCORED BIO LTD., REHOVOT (IL)

(21) Appl. No.: 12/843,958

(22) Filed: Jul. 27, 2010

Related U.S. Application Data
(62) Division of application No. 10/587,456, filed on May 21, 2007.

(30) Foreign Application Priority Data
Jan. 28, 2004 (IL) 160095

(51) Int. Cl. A61K 9/66; A61K 9/28; A61K 9/14; A61K 9/50; A61K 9/107

(52) U.S. Cl. 424/455; 424/474; 424/490

(57) ABSTRACT

The present invention provides a drug delivery system comprising nanoparticles or microparticles of a water poorly soluble drug dispersed in a polymeric bead containing essentially only of hydrophilic polymers (i.e. without hydrophobic polymers). The present invention further provides a method of producing the drug delivery system of the invention.

### FIELD OF THE INVENTION

The present invention generally concerns formulations for drugs, and more particularly formulations for poorly soluble drugs.

### BACKGROUND OF THE INVENTION

Solubility is defined as the concentration of the solute in a saturated solution. The solubility of compounds varies in accordance with factors such as temperature, the type of solvent, the pH of the solution, and atmospheric pressure. The solubility of drugs found in the US Pharmacopeia is expressed as the number of milliliters of solvent in which one grain of solute can dissolve. Where the exact solubility of various compounds cannot be precisely determined general quality terms are used to describe the solubility of a specific compound, typically with reference to other compounds. Solubility may also be expressed in terms of molarity, percentage, and molality. Typically, drugs defined as “poorly soluble” are those that require more than 1 ml part of solvent per 10 mg of solute. Some poorly soluble drugs are further limited by their intrinsic bioavailability for example due to extensive first pass metabolism by the liverok (first pass effect), or further limited due to various drug-drug interactions.

Usage of poorly soluble compounds has increased by 25% on average over the last five year period. The increase in formulations containing poorly soluble compounds is attributed to factors associated with both the pharmaceutical and biotechnology sectors. For example, within the pharmaceutical sector, drugs are now more frequently designed by combinatorial chemistry in order to improve their distribution through various tissues in the body, increase their half life, and improve their therapeutic index (more potency with low concentrations). Sometimes newly developed drugs produced by combinatorial techniques are poorly soluble as during development, and in contrast to rational drug design, solubility was never a factor considered for their production.

In the biotechnology field, compounds, such as peptides, nucleic acid sequences, monoclonal antibodies, etc. resulting from biotechnological development are also typically poorly soluble.

There are several different approaches to solve the problem of solubility of poorly soluble drugs. These include traditional solubilizing approaches using a combination of solvents, surfactants and co-solvents, various sophisticated dispersion systems, as well as novel technologies, including micronization, complexation and liposomal delivery.

One approach directed to delivery and release of poorly soluble drugs is their formulation as nano sized particles/crystals.

U.S. Patent Application 20030215513 concerns release of substantially water insoluble nano-sized particles from a composition, by coating the pharmaceutical composition with a diffusion-control membranes that contains a multiplicity of pores and pore-forming substances. This establishes a diffusion gradient that enables mass-transport of nano-suspensions from the pharmaceutical composition through the pores, thereby resulting in a diffusion controlled release through the membrane.

U.S. Patent Application 20020106403 discloses a water insoluble drug, in a nanometer or micrometer particulate solid format, which is surface stabilized by a phospholipid, being dispersed throughout a bulking matrix. This construction can dissolve, upon contact with aqueous environments, thereby releasing the water insoluble particulate solid in an unaggregated or un-agglomerated form. Typically, the matrix is composed of water insoluble substance.

U.S. Pat. No. 5,439,686 discloses compositions for in vivo delivery of water insoluble pharmaceutical agents, notably the anticancer drug taxol, wherein the active agent is solubilized in a biocompatible dispersing agent contained within a protein walled shell. By another alternative, the protein walled shell can contain particles of the taxol itself.

U.S. Pat. No. 6,387,409 discloses nano- or micro-sized particles of water insoluble, or of poorly soluble drugs, produced by a combination of natural and synthetic phospholipids and charge surface modifiers such as highly purified charge phospholipids, together with a block copolymer which are coated or adhered on to the surfaces of water insoluble compound particles. These constructs enable the formation and stabilization of submicron and micron sized compound particles stabilized by the charge phospholipids which provides electrostatic stabilization; and stabilized by the block copolymer to provide steric stabilization. Such constructs prevent the particles from aggregation and flocculation.

International Patent Application WO 9725028 concerns controlled release beads which comprise a core of insoluble drugs, and a layer of furosemide dispersed in a hydrophilic polymer and a membrane which regulates the release of the furosemide in a controlled manner.

U.S. Pat. No. 6,645,528 concerns poorly soluble drugs provided in a porous matrix form which enhances the dissolution of the drug in an aqueous media. The pore forming agent creating the porous matrix is typically a volatile liquid that is immiscible with the drug solvent, or alternatively, a volatile solid compound such as a volatile salt. The resulting porous matrix has a faster rate of dissolution following administration to a patient as compared to a non porous matrix form of the drug.

Sustained, or controlled release drug delivery systems, include any drug delivery system that achieves a slow release of a drug over an extended period of time. The main aim of slow release systems is improved efficiency of treatment as a result of obtaining constant drug-blood levels, thus maintaining the desired therapeutic effect for extended periods of time. This results in reduction and elimination of fluctuations in blood levels, thus allowing better disease management.

Some controlled release systems were not developed for the main purpose of sustained release, but rather having been developed in order to improve the bioavailability of drugs, due to their activity in isolating the drugs from the environment, for example by protecting drugs susceptible to enzymatic inactivation or bacterial decomposition by encapsulation in polymeric systems.

Microparticles containing poorly soluble drugs and a polymer were prepared in order to overcome some technical problems of tabulating encountered during formulations of medicaments with microparticles. In these formulations propranonol was the poorly soluble drug, and the polymer was ethylcellulose. Together, the polymer and the poorly soluble drugs were mixed to form microspheres containing a drug-polymer mixture, which were subsequently entrapped within a chitosan or calcium alginate beads. Thus the beads contained initially a mixture of drugs and insoluble polymers, subsequently mixed with a soluble polymer. The ionic characteristics of the polysaccharides of this delivery system allowed a pH-dependent release of the microparticles in the gastrointestinal tract (Bodmeier et al. Pharmaceutical Research 6:5, 1989).

### SUMMARY OF THE INVENTION

The present invention is based on the realization that particles of water insoluble or poorly soluble drugs can have improved solubility, and hence improved bioavailability, if they are administered dispersed in a hydrophilic polymeric bead in the form of nanoparticles or microparticles of the drug.

Thus, by one aspect the present invention concerns a drug delivery system comprising nanoparticles or microparticles of a poorly soluble drug dispersed in a polymeric bead containing essentially only of hydrophilic polymers (i.e. without hydrophobic polymers).

The term “nanoparticle” in the context of the drugs refers to particles which have the size of 3 nm to 900 nm, preferably 5 nm to 450 nm. Similarly, the term “microparticle ” refers to particles which have the size of 1 to 500 micrometers.

By a preferred embodiment, the polymeric beads consist essentially of a single hydrophilic polymer, this being in contrast to the publication of Bodmeier et al. wherein the poorly soluble drug is first entrapped within an insoluble, hydrophobic polymer, and the obtained microparticles of the insoluble polymer and drug are then mixed with a soluble polymer-forming bead. Therefore, by Bodmeier publication one obtains drug molecules entrapped within a water insoluble polymeric matrix, which leads to decreased solubility of the drug, and that would cause a decreased bioavailability.

Against this, the beads of the present invention consist of drug nanoparticles essentially free of water insoluble polymer, while the single hydrophilic polymer serves as a former of porous bead, which prevents the increase in the size of the drug particle, and greatly simplifies the manner of production as will be explained hereinbelow.

In addition, in accordance with one preferred embodiment of the invention, the bead formation process by itself leads to formation of the drug nanoparticles, which are formed from a nanoemulsion, in a way that overcomes the problems associated with conventional methods for preparation of nanoparticles by solvent evaporation from submicron emulsions. The beads themselves serve as the delivery system, having the ability of controlling the release of the nano/micro particles of the poorly soluble drugs therefrom. The control can be achieved by the inherent polymeric structure of the bead, or by a combination of the bead skeleton polymers and polymeric additives, mainly water soluble polymers.

The term “drug delivery system” in the context of the present invention concerns active ingredient—i.e. the drug—in its carrier matrix. The drug delivery system in accordance with the invention may be used for subsequent preparation of dosage administration forms, for example, in the form of capsules (coated or uncoated), tablets (coated or uncoated), wherein the coating may be functional such as enteric coating, colonic delivery coating, chrono-therapeutic and controlled release coating, taste-masking coating and the like. The dosage form may be suitable for any mode of administration such as oral, rectal, depo-administration, parenteral, subcutaneous, ocular, nasal, vaginal and the like.

The term “polymer” in accordance with the present invention shall be understood as referring both to a polymer composed of a single re-occurring building block (monomer) as well as to a polymer composed of two or more different polymeric units (co-polymer).

The term “poorly soluble drug” refers to a drug which is insoluble or poorly soluble in an aqueous solution, and typically this refers to a drug which has a solubility of less than 10 mg/ml, and preferably less than about 5 mg/ml in aqueous media at approximately physiological temperature and pH. As used herein, the term “drug” refers to chemical and biological molecules having therapeutic, diagnostic or prophylactic effects in vivo. The term “drug” therefore may include food additives which have biological activity such as lycomene, lycopene and beta carotene.

Drugs contemplated for use in the system described herein include the following categories and examples of drugs and alternative forms of these drugs such as alternative salt forms, free acid forms, free base forms, prodrug forms and solvates e.g. hydrates: Accupril (Quinapril), Accutane (Isotretinoin), Actos (Pioglitazone), AeroBid (Flunisolide), Agenerase (Amprenavir), Akinetron (Biperiden), Allegra (Fexofenadine), Aromasin (Exernestane), Asacol (Mesalamine), Atacand (Candesartan cilexetil), Avandia (Rosiglitazone), Azmacort (Triamcinolone), Biaxin (Clarithromycin), Camptosar (Irinotecan), Cefzon (Cfdinir), Celebrex (Celecoxib), Claritin (Loratadine), Clinoril (Sulindac), Cordarone (Amiodarone HCL), Diovan (Valsartan), Duragesic (Fentanyl citrate), DynaCirc (Isradapine), Elmiron (Pentosan polysulfate sodium), Elconon/Nasonex (Mometasone), Epogen/Procrit (EPO), Estratest (Methyltestosterone), Evista (Raloxifene hydrochloride), Fareston (Toremifene citrate), Flomax (Tamsulosin hydrochloride), Follistirn (Follitropin beta), Halcion (Triazolam), Hismanal (Astemizole), Hydergine LC (Ergoloid mesylates), Imodium (Loperamide), Invirase (Saquinavir), Lipitor (Atorvastatin Calcium), Luvox (Fluvoxamine), Mevacor (Lovastatin), Neoral and Sandimmune (Cyclosporine), Nitorol-R/Frandol (Isosorbide dinitrate), Noroxin (Norfloxacin), Norvir (Ritonavir), Pepcid (Famotidine), Platinol-AQ (Cisplatin), Plavix (Clopidrogel bisulfate), Plendil (Felodipine), Pletal (Cilostazol), Pulmicort Turbuhaler/Rhinocort (Budesonide).

The drugs may also include biological produced agents.such as proteins, protein fragments, peptides, nucleic acid sequences, oligonucleotides, glycoproteins as long as they are water insoluble

Most preferable drugs are simvastatine, statines, risperidone, carvedilol, carbamazepine, oxcarbazepine, zaleplon, galantamine, avandia, and poorly soluble anti psychotic, anti epileptic, anti parkinsonian and other indicated for CNS indications.

The polymeric bead may comprise at least one of a polysaccharide polymer, a protein, a synthetic polymer which may be either crosslinked or not crosslinked or mixtures thereof.

Examples of polysaccharide polymers are: alginates, chitosans, gellan gums, agarose, pectin, carrageenan.

Examples of proteins are: gelatins, albumins, lactalbumin.

Examples of synthetic polymers are polyacrylic acid, polyethylene glycol (“PEG”), polyvinyl pyrrolidone, polymethacrylates, polylysine, poloxamers, polyvinyl alcohol, polyethylene oxide, and polyethyoxazoline.

Preferably, in accordance with the present invention, the nanoparticles or microparticles are in an amorphous state, which increases their solubility rate, and subsequent crystallization is prevented due to the presence of hydrophilic polymer and surfactants used in the process of production.

Still more preferably, in accordance with the invention, the drug delivery system may include externally added crosslinking agents, which are, for anionic polyssacharides and synthetic polymers, multivalent cations, such as calcium, magnesium, barium, ferrous, polycations and cupper salts. For cationic polymers, such as chitosan, a polyvalent anion such as tripolyphosphate or anionic polymers may be used. It sould be noted that the polymeric beads may also be formed by heating-cooling effects, such as formation of gelatin beads, which is obtained by dropwise addition of warm gelation solution into cold liquid, water or oil.

Still more preferably, the drug delivery system including said externally added crosslinking agents, further comprises a disintegrant which may be a chelator of the crosslinking cation, for example calcium or magnesium. Such chelators, in contact with water, interact with the crosslinking agents, thus breaking the crosslinking of the polymeric bead and enhancing the disintegration of the bead.

Examples of disintegrants are EDTA, sodium citrate, citric acid, sodium dodecyl sulfate, phosphate salts and phosphate buffer saline. By using a disintegrate mixed with the polymer bead in the delivery system of the invention, it is possible on the one hand to improve the solubility of the poorly soluble drugs by using the drug in the form of nanoparticles, and on the other hand to obtain rapid disintegration of the bead, for example in the gastrointestinal tract, in such a way that the drug nanoparticles are in close contact with the dissolution medium, without any barrier that could be formed by the crosslinked polymer.

Such a construct which is unusual for polymeric beads, which typically are constructed without a disintegrant for sustained-release purposes, which results in drug particles that remain entrapped in the beads' core leading to slower dissolution rate and consequently to reduced bioavailability.

Thus the present invention concerns a drug delivery system comprising an active ingredient dispersed within a polymeric bead, wherein the polymer may be crosslinked, while the crosslinking is achieved (in case of sodium alginate, for example) by a multivalent cation such as calcium, magnesium, barium, ferrous or copper salts and wherein the drug delivery system further comprises as a disintegrate, a chelator of the multivalent cation.

Preferably, the drug is a poorly soluble drug, more preferably in the form of a nano-particle, a micro-particle, most preferably in the form of a nanoparticle.

The present invention further concerns a method of producing the drug delivery system of the invention comprising:
(i) providing poorly water soluble drug dissolved in organic volatile solvent, optionally in the presence of at least one surfactant;
(ii) mixing the drug-containing solvent with an aqueous phase, optionally in the presence of at least one agent selected from surfactant, co-solvent and electrolyte, thereby producing an oil-in-water nanoemulsion or rnicroemulsion;
(iii) mixing the oil-in-water nano- or micro emulsion with water-soluble bead-forming polymers to produce a continuous phase of the emulsion which comprises the bead forming polymer;
(iv) providing conditions enabling bead formation from the continuous phase of (iii);
(v) drying of the beads, by evaporating the volatile organic solvent and the aqueous phase of the bead;

thereby obtaining dry beads comprising in their matrix dispersed nanoparticles or microparticles of poorly water-soluble drugs.

The beads containing the drug nanoparticles or microparticles obtained by the method of the invention may be formulated to fonn a suitable dosage form, for example they may be packed within a capsule or a tablet, optionally together with a disintegrant as will be explained herein bellow, thus providing a delivery system of the poorly soluble drug. Alternatively polymeric additives may be added in order to control the drug release.

The poorly soluble drug is rendered in a nanoparticle form by consequent evaporation of the organic solvent and the water, thus the previously dissolved drug in the solvent droplets, becomes insoluble, and having a size similar to the initial size of the nanoemulsion droplets, and in most cases having a non-crystalline morphology. Since each nanoemulsion droplet is dispersed within the crosslined polymeric network of the bead, there is no possibility for coalescence of emulsion droplets, and therefore there is no increase in the size of drug particles which are maintained in their original nanoparticle size. In addition, since the evaporation of the solvent is rapid, and performed within a viscous, crosslinked polymeric network (which becomes more viscous as evaporation proceeds), the obtained drug nanoparticles are amorphous (not crystalline).

Furthermore, due to the presence of the surfactants in the nanoemulsion the nanoparticles remain in an amorphic structure that brings significant advantages for enhanced dissolution and bioavailability.

As will be shown in the examples, the processes described in this invention allow obtaining nanoparticles of drugs, which otherwise, upon application of conventional solvent evaporation method, would have formed large crystals. It was surprisingly found that by performing the solvent evaporation process only after the beads are formed, the crystallization and increase of the size of the drug molecule could be prevented.

The solvent used in the method of the invention is an organic solvent that is volatile (at the concentration used) i.e. has a relatively low boiling point, or can be removed under vacuum, and which is acceptable for administration to humans in trace amounts. Representative solvents include, chloroform, chlorofluorocarbons, dichloromethane, dipropyl ether, diisopropyl ether, ethyl acetate, butyl acetate, methyl ethyl ketone (MEK), limonene, heptane, hexane, butanol, octane, pentane, toluene, 1,1,1-trichloroethane, 1,1,2-trichloroethylene, xylene, and combinations thereof. In general, the drug is dissolved in the volatile solvent to form a drug solution having a concentration of between 0.01 and 80% weight to volume (w/v). Alternatively, the solvent in which the drug is dissolved may contain a co-solvent which is either miscible or immiscible with water. Examples for co-solvents are: ethanol, isopropanol, pentanol THF, DMF, DMSO, propylene glycol, polyethylene glycol, glyme diglyme, triglyme and the like.

Examples of suitable surfactants are: nonionic surfactants such as for example block copolymers, e.g. Pluronic F 68, polyglycerol esters, alkyl glucosides ethoxylated sorbitan esters and ethoxylated sorbitan esters; ionic surfactants; and polymers such as polyvinyl alcohol, gelatin and BSA.

The surfactants are selected from molecules acceptable for pharmaceutical preparations, which are capable of yielding nanoemulsions or microemulsions. The nanoemulsions can be formed by various methods, preferably by using a high pressure homogenization technology, or phase inversion methods (such as the PIT method) and the microemulsions are prepared by simple mixing of proper compositions of water, surfactants, solvents and co-solvents (microemulsions may form spontaneously, according the phase diagram of the compositions).

Additional exemplary surfactants which may be used include most physiologically acceptable emulsifiers, for instance egg lecithin or soya bean lecithin, or synthetic lecithins such as saturated synthetic lecithins, for example, dimyristoyl phosphatidyl choline, dipalmitoyl phosphatidyl choline or distearoyl phosphatidyl choline or unsaturated synthetic lecithins, such as dioleyl phosphatidyl choline or dilinoleyl phosphatidyl choline. Surfactants also include salts of fatty acids, esters of fatty acids with polyoxyalkylene compounds like polyoxpropylene glycol and polyoxyethylene glycol; ethers of fatty alcohols with polyoxyalkylene glycols; esters of fatty acids with polyoxyalkylated sorbitan; soaps; glycerol-polyalkylene stearate; glycerol-polyoxyethylene ricinoleate; homo- and co-polymers of polyalkylene glycols; polyethoxylated soya-oil and castor oil as well as hydrogenated derivatives; ethers and esters of sucrose or other carbohydrates with fatty acids, fatty alcohols, these being optionally polyoxyalkylated; mono-, di- and tri-glycerides of saturated or unsaturated fatty acids, glycerides of soya-oil and sucrose.

Beads are formed by solidifying drops of solutions containing the bead forming polymers either by contact with a crosslinking agent (when the polymer can react with the crosslinking agent to form an insoluble polymeric structure), or by solidification, for examples while using a polymer such as gelatin, which forms a liquid solution at elevated temperature, and solidifies at room temperature.

Thus, while the bead forming solution is added as small droplets through a suitable orifice, into a crosslinking solution or simply in a cold environment in case of temperature induced bead formation, immediate crosslinking (similar to solidification) of the external part of the bead occurs, and therefore the external part of the droplets becomes solid.

Upon further exposure to the crosslinking solution, the crosslinking ions migrate into the interior part of the bead, and form a solid matrix throughout the whole bead.

The structure of the beads (porosity, rigidity etc.) can be tailored by proper selection of the bead formation conditions (such as crosslinker concentration, duration of crosslinking, presence of various electrolytes etc.). The size of the beads can be controlled by proper selection of the nozzle diameter and instrumentation from which the bead forming polymeric solution is ejected.

Finally, as a last stage, the volatile (organic solvent) is evaporated together with the aqueous phase, for example by application of vacuum or by lyophilization processes, or by simply drying at room temperature or in an oven at elevated temperatures, to obtain the dry beads containing in their matrix dispersed nanoparticles of the poorly soluble drug.

At the last preparation step, the beads are packed in a suitable pharmaceutical formulation such as gelatin capsule or solid tablet (containing conventional pharmaceutical excipients), and optionally containing agents which enhance the disintegration of the beads upon contact with body fluids. Such disintegrators can be molecules capable of replacing the crosslinking agent, such as chelators of the crosslinking agents such as EDTA, citric acid, sodium citrate, or surfactants such as sodium dodecyl sulfate, phosphate salts or phosphate buffer saline.

Thus, when the polymeric beads are placed in an aqueous medium (such as in the gastrointestinal tract) water activates the disintegrating agent, causing it to chelate (for example in case the disintegrant is a chelator) the crosslinkers (such as calcium ions), thereby disintegrating the beads and speeding up the release of the drug therefrom. Agents which modify the release, such as polymers may be added to the pharmaceutical dosage forms as well for decreasing rather then increasing, the release rate.

Polymeric bead properties can be tailored to meet various requirements for proper drug dissolution as will be explained below.

### BRIEF DESCRIPTION OF THE DRAWINGS

In order to understand the invention and to see how it may be carried out in practice, some preferred embodiments will now be described, by way of non-limiting examples only, with reference to the accompanying drawings, in which:

FIG. 1A shows an electron microscope picture of a polymeric bead containing nanoparticles of simvastatine, prepared as described in Example 1 which are vacuum dried;

FIG. 1B shows an electron microscope picture of a cross section of the polymeric bead shown in FIG. 1A.

FIG. 1C shows an electron microscope picture of a polymeric bead containing nanoparticles of simvastatine, prepared as described in Example 1 which are air dried.

FIG. 1D shows an electron microscope picture of a cross section of the polymeric bead shown in FIG. 1C.

FIG. 2 shows the dissolution of two samples of beads of the invention containing simvastatine as compared to dissolution of commercial simvastatine.

FIG. 3 shows an electron microscope picture of simvastatin crystals after solvent evaporation carried out without using bead formation.

FIG. 4 shows electron microscope pictures of simvastatin nanoparticles after solvent evaporation from bead nanoemulsion systems.

FIG. 5 shows the effect of varying concentrations of phosphate buffer (pH ˜6.8) on beads disintegration.

FIG. 6 shows the effect of varying concentrations of citrate buffer (pH ˜6.8) on beads disintegration.

FIG. 7 shows the effect of various crosslinking ions at a concentration of 25 mM on beads disintegration.

FIG. 8 shows the effect of various crosslinking ions at a concentration of 100 mM on beads disintegration.

### DETAILED DESCRIPTION OF THE INVENTION

### Tailoring of the Polymeric Bead Parameters

The following parameters may be varied when designing the drug delivery system of the present invention:
1) Droplets size in the nano/microemulsion may be tailored by controlling volatile solvent type, co-solvent type, surfactants and co-surfactant concentration and type, by controlling the cycles in high-pressure homogenizer (in case high pressure homogenization is utilized to obtain the nanoemulsions), o/w ratio and temperature.
2) Type and molecular weight of the polysaccharide, (e.g. Alginate, K-Carrageenan, Chitosan, Gellan gum, Agarose, Pectin etc,) or synthetic polymers.
3) Structure of alginates (e.g. different ratio of guluronic and mannuronic acids).
4) Type and concentration of the crosslinking agent (also termed “gelling agent”) ion solution (cation: Ca⁺² , Ba⁺² , AL⁺³ ,Fe⁺², Cu⁺² , poly(amino acids) etc., and non-crosslinking ion (and Na⁺).
5) Crosslinking duration.
6) Matrix composition of material other than the bead forming polymer: other materials may be added, such as Silica, HPMC, Lactose, sodium chloride etc., which affect the morphology, porosity, size, and shrinkage of beads upon drying, disintegration rate and hydrophobicity.
7) The size of the polysaccharide beads can be controlled by controlling nozzle size, frequency, amplitude, velocity, physical parameters.
8) The rate of disintegration may be controlled by adding a disintegrate such as EDTA, phosphate or citrate ions, and controlling the amount of the disintegrant.

### EXAMPLE 1

### Solutions Preparation

### 4% Alginate Solution

16 g of Alginic acid sodium salt (Sigma, low viscosity, 2% solution-250 cps) was dissolved in 400 g distilled water (4% w/w), together with 0.4 g of Bronopol (preserving material). The mixture was mixed on magnetic stirrer for about 48 hours and heated to about 37° C. until complete dissolution.

### 100 mM CaCl, Solution (Crosslinking Agent)

14.8 g of Dihydrate Calcium Chloride (Merck) was dissolved in 1000 g distilled water.

### 1. Emulsification

Oil in water emulsion 20% oil phase fraction, 80% aqueous phase fraction was prepared, containing 3% w/w total surfactant (mixture of Tween 20, commercial name ofethoxylated sorbitan mono-laurate and Span 20, commercial name ofsorbitan monolaurate HLB=10) concentration.

3.3584 g of Simvastatine powder (Teva Pharmaceuticals, Israel) used as the poorly soluble drug was weighed and mixed with 80.0 g toluene until complete dissolution of the drug is achieved. Final concentration of Simvastatine is 42 mg/g toluene.

1.02 g Tween 20 was weighed and dissolved in 160.26 g distilled water saturated with toluene (filtered through 0.2 μm filter).

4.97 g Span 20 was weighed and mixed with the 40.23 g solution of 42 mg/g Simvastatine in toluene, and stirred about 10 min together. The organic phase was added carefully to the water phase and mixed for 5 min in an Ultra Turrax homogenizer at 8000 RPM. A coarse, homogeneous emulsion was obtained. This emulsion was introduced into a high pressure homogenizer (Stansted), and was circulated through the high-pressure-homogenizer twice at 17,000 psi.

Z-average particles size of the resulting emulsion was 250-255 nm.

### 2. Beads Formation

95.1 g of sodium alginate solution (4% w/w) and 3.8 g of Silica 60 Å Frutarom) used to prevent shrinking upon drying, were mixed together for about 10 min by a magnetic stirrer until the silica was dispersed homogeneously in the alginate solution. Then 95.1 g of the above o/w emulsion were added and stirred together until homogenous mixture was achieved. The alginate-emulsion mixture was introduced into an Innotech encapsulator, and jetted into 100 mM CaCl₂ crosslinking solution.

The Innotech encapsulator allows tailoring the final size of the beads by selecting the proper instrument parameters. In this example, the parameters were:

Nozzle size—300 μm.

Voltage—0.914 Kv.

Amplitude—3.

Frequency—1550 Hz.

Pressure—0.4 bar.

The beads were kept in the crosslinking solution for 30 min.

Then, the beads were rinsed with about 2 liters of distilled water, filtered and air dried in an oven, at temperature of about 35° C. for 48 hours, in order to remove the water and the volatile solvent.

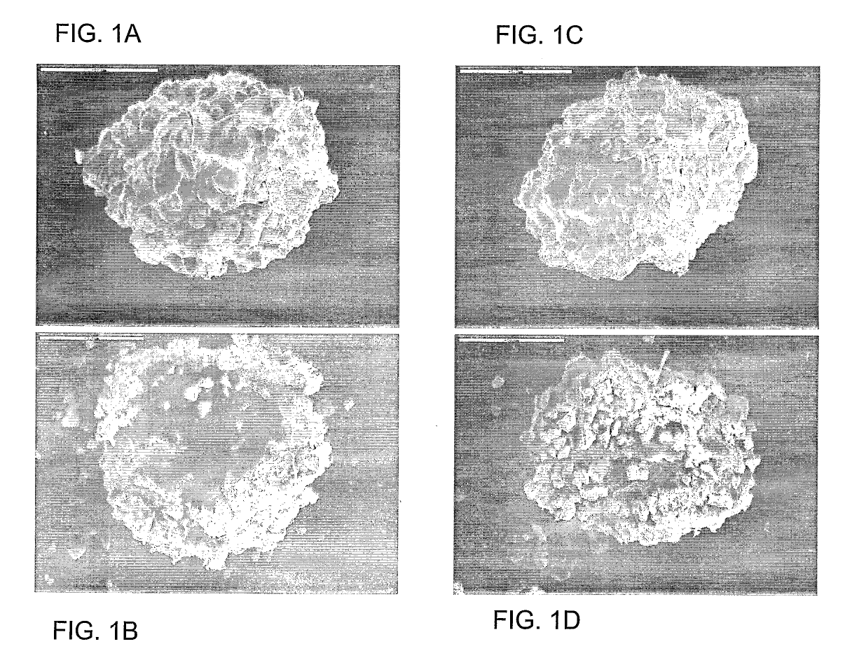

The final result was dry beads in the size range of less than 1 mm in which nanoparticles of Simvastatine were dispersed, as verified by electron microscopy and shown in FIG. 1. FIG. 1A shows an electron microscope picture of a polymeric bead containing nanoparticles of simvastatine, which was vacuum dried. A cross section of same bead is shown in FIG. 1B. FIG. 1C shows an electron microscope picture of a polymeric bead containing nanoparticles of simvastatine, which was air dried. A cross section of same bead is shown in FIG. 1D.

### EXAMPLE 2

### Reduction of Gelling Time and Gelling Ion Concentration. Solutions Preparations

### 4% Alginate Solution: Was Prepared as Described in Example 1.

### 25 mM CaCl, Solution (Crosslinking Agent)

3.7 g of Dihydrate Calcium Chloride (Merck) was dissolved in 1000 g distilled water.

### 1. Emulsification

Oil in water emulsion 20% oil phase fraction, 80% aqeous phase fraction was prepared, containing 3% w/w total surfactant (mixture of Tween 20 and Span 20, HLB=10) concentration. 3.7869 g of Simvastatine powder (Teva Pharmaceuticals, Israel) used as the poorly soluble drug was weighed and mixed with 90.1 g toluene until complete dissolution of the drug is achieved. Final concentration of Simvastatine is 42 mg/g toluene.

1.04 g Tween 20 was weighed and dissolved in 160.54 g distilled water saturated with toluene (filtered through 0.2 μm filter).

4.97 g span 20 was weighed and mixed with the 40.55 g solution of 42 mg/g Simvastatine in toluene, and stirred about 10 min together. The organic phase was added carefully to the water phase and mixed for 5 min in an Ultra Turrax homogenizer at 8000 RPM. A coarse, homogeneous emulsion was obtained. This emulsion was introduced into a high pressure homogenizer (Stansted), and was circulated through the high-pressure-homogenizer twice at 17,000 psi.

Z-average particles size of the resulting emulsion was 194-210 nm.

### 2. Beads Formation

75.3 g of sodium alginate solution (4% w/w) and 3.0 g of silica 60 Å (Frutarom) were mixed together for about 10 min by a magnetic stirrer until the silica was dispersed homogeneously in the alginate solution. Then 75.2 g of the above o/w emulsion were added and stirred together until homogenous mixture was achieved. The alginate-emulsion mixture was introduced into an Innotech encapsulator, and jetted into 25 mM CaCl₂ crosslinking solution.

The Innotech encapsulator allows tailoring the final size of the beads by selecting the proper instrument parameters. In this example, the parameters were:

Nozzle size—300 μm.

Voltage—1.005 Kv.

Amplitude—3.

Frequency—1527 Hz.

Pressure—0.3 bar.

The beads were kept in the crosslinking solution for 10 min.

Then, the beads were rinsed with about 2 liters of distilled water, filtered and air dried in an oven, at temperature of about 35° C. for 48 hours, in order to remove the water and the volatile solvent.

### EXAMPLE 3

### Alteration of Surfactant

### Solutions Preparations

### 4% Alginate Solution

Was prepared as described in Example 1.

### 25 mM CaCl₂ Solution (Crosslinking Agent)

Was prepared as described in Example 2.

### 1. Emulsification

Oil in water emulsion 20% oil phase fraction, 80% aqeous phase fraction was prepared, containing 3% (w/w) total surfactant (Hexaglycerol sesquistearate, SY-GLYS SS-5S, SAKAMOTO YAKUHIN KOGYO CO., LTD. HLB=9.9) concentration. 3.7807 g of Simvastatine powder (Teva Pharmaceuticals, Israel), used as the poorly soluble drug was weighed and mixed with 90.1 g toluene until complete dissolution of the drug is achieved. Final concentration of Simvastatine is 42 mg/g toluene.

4.02 g Hexaglycerol sesquistearate was weighed and dissolved in 160.28 g distilled water saturated with toluene (filtered through 0.2 μm filter).

2.02 g Hexaglycerol sesquistearate was weighed and mixed with the 40.46 g solution of 42 mg/g Simvastatine in toluene, and stirred about 10 min together. The organic phase was added carefully to the water phase and mixed for 5 min in an Ultra Turrax homogenizer at 8000 RPM. A coarse, homogeneous emulsion was obtained. This emulsion was introduced into a high-pressure homogenizer (Stansted), and was circulated through the high-pressure-homogenizer twice at 17,000 psi.

Z-average particles size of the resulting emulsion was 126-140 nm.

2. Beads Formation

75.2 g of sodium alginate solution (4% w/w) and 3.0 g of Silica 60 Å (Frutarom) were mixed together for about 10 min by a magnetic stirrer until the silica was dispersed homogeneously in the alginate solution. Then 75.5 g of the above o/w emulsion were added and stirred together until homogenous mixture was achieved. The alginate-emulsion mixture was introduced into an Innotech encapsulator, and jetted into 25 mM CaCl₂ crosslinking solution.

The Innotech encapsulator allows tailoring the final size of the beads by selecting the proper instrument parameters. In this example, the parameters were:

Nozzle size—300 μm.

Voltage—1.005 Kv.

Amplitude—3.

Frequency—1527 Hz.

Pressure—0.3 bar.

The beads were kept in the crosslinking solution for 10 min.

Then, the beads were rinsed with about 2 liters of distilled water, filtered and air dried in an oven, at temperature of about 35° C. for 48 hours, in order to remove the water and the volatile solvent.

### Dissolution Tests

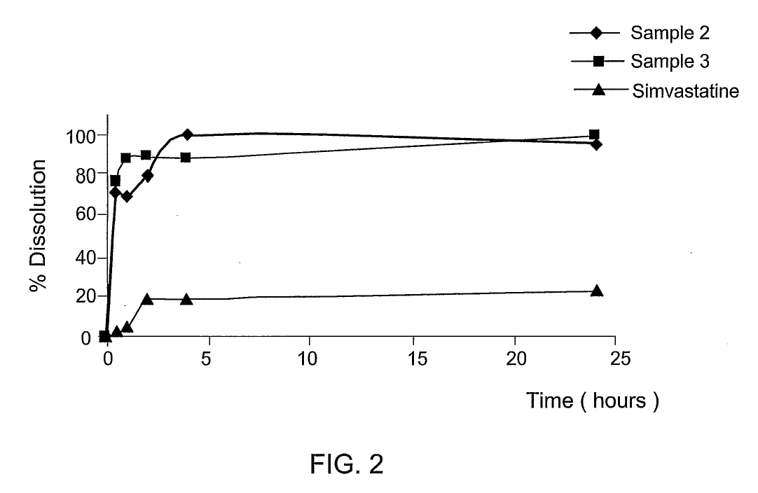

Dissolution test was performed to the dried beads and the results are shown in FIG. 2, where samples 2 and 3 are the beads of the invention compared to commercial simvastatine.

Dissolution test parameters:

Instrument: Caleva 7ST , Test method: USP II at 75 rpm

Dissolution medium: Citarate Buffer 0.1 M pH-6.8

Assay Procedure: UV at 239 nm.

Dissolution test shows (see FIG. 2) the advantage of the beads of the invention, which uses hydrophilic polymer beads containing dispersed nano-particles of simvastatine (water insoluble drug) by solvent evaporation upon commercial simvastatine particles.

The overall dissolution rate of the beads containing dispersed nanoparticles is much faster than that of commercial drug particles. Using beads nanoparticles system enable tailoring of release kinetics.

The dried resulting beads can be inserted to capsules or compressed to tablets.

### EXAMPLE 4

### Solvent Evaporation of Nanoemulsion in Conventional Way

In this example solvent evaporation was performed to the nanoemulsion before beads formation. This experiment prove the necessity of solvent evaporation after the beads formation in order to prevent crystal formation and growing of the lipophilic drug.

### 1. Emulsification

Oil in water emulsion 20% oil phase fraction, 80% aqueous phase fraction was prepared, containing 3% (w/w) total surfactant (mixture of Tween 20 and Span 20, HLB=10) concentration. 2.5231 g of Simvastatine powder (Teva Pharmaceuticals, Israel) used as the poorly soluble drug was weighed and mixed with 61.7 g toluene until complete dissolution of the drug is achieved. Final concentration of Simvastatine is 41 mg/g toluene.

0.51 g Tween 20 was weighed and dissolved in 80.26 g distilled water saturated with toluene (filtered through 0.2 μm filter).

2.49 g Span 20 was weighed and mixed with the 20.56 g solution of 41 mg/g Simvastatine in toluene, and stirred about 10 min together. The organic phase was added carefully to the water phase and mixed for 5 min in an Ultra Turrax homogenizer at 8000 RPM. A coarse, homogeneous emulsion was obtained. This emulsion was introduced into a high pressure homogenizer (Stansted), and was circulated through the high-pressure-homogenizer twice at 17,000 psi.

Z-average particles size of the resulting emulsion was 186-198 nm.

The organic solvent (toluene) was evaporated with Rotavapor (R-114 BUCHI) from the emulsion to form a dispersion of lipophilic drug in water. The organic solvent evaporation was performed in four steps, water was added up to the initial weight after each step.

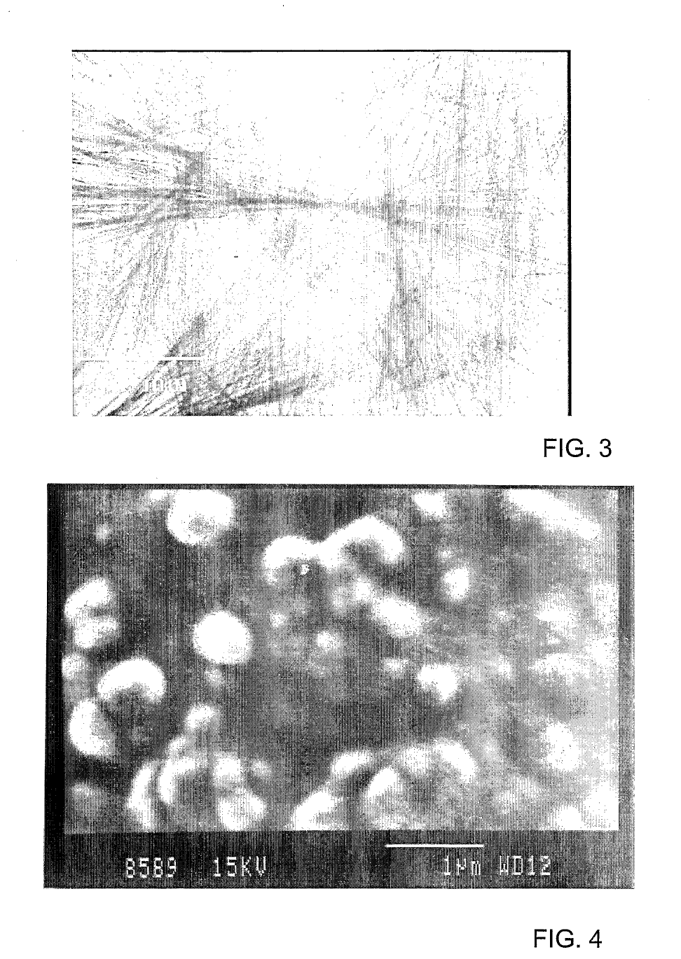

After several hours, it was found that huge large crystals (needles) (crystal size: 0.5-2 mm) of the raw material were formed (see FIG. 3) that indicate the instability of the drug nanoparticles that was formed after evaporation, while the evaporation is performed not within the polymeric bead.

Against this, when the solvent evaporation was performed after the beads formation, the simvastatine remain as nanoparticles while performing the evaporation without beads forms large crystals of simvastatine (see FIG. 4). These experiments prove the necessity of solvent evaporation after the beads formation in order to prevent forming and growing of the drug crystals, which significantly reduce the bioavailability of the poorly soluble drug.

### EXAMPLE 5

### Disintegrant Effect on the Beads

Alginate beads are insoluble in water or acidic media. In order to enable the disintegration of the drug uptake, a disintegrant was included in the drug formulation, which contains the beads. The effect of disintegrant is demonstrated by experiments in which the beads were immersed in liquid containing the disintegrant.

The beads disintegration measurements were performed using turbidimeter (HACH RATIO/XR). The turbidity values represent the beads disintegration. It is expected that the disintegration will enhance the drug release in the system. It should be emphasize that the beads cannot disintegrate without the presence of suitable disintegrating agents.

FIG. 5 demonstrates the influence of phosphate buffer concentrations, in the range of 0.05 M-0.25 M, on the beads disintegration rate. In 0.05 M phosphate buffer the beads were slightly disintegrated while in 0.25 M phosphate buffer the beads were completely disintegrated within 10 mins.

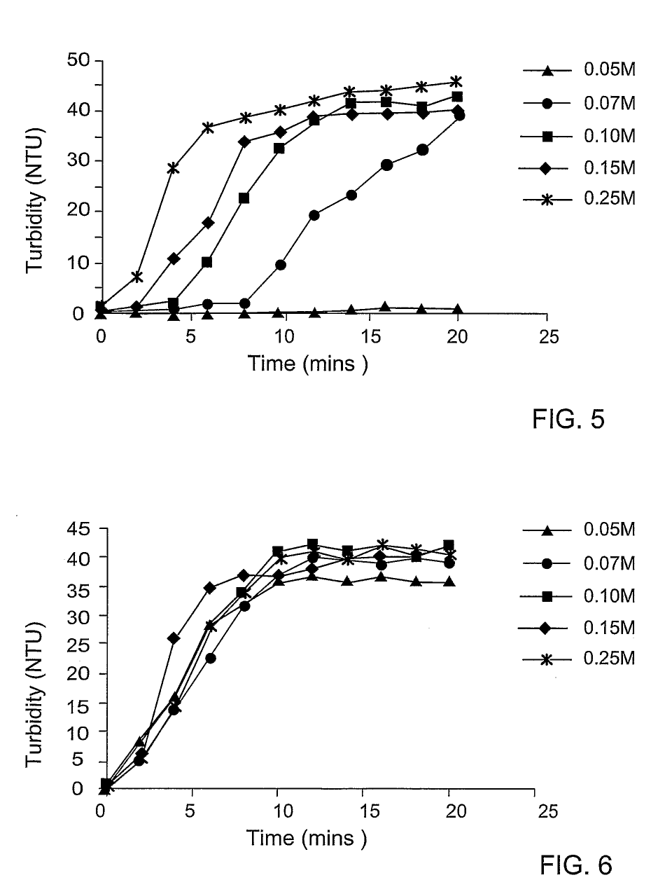

FIG. 6 demonstrates the influence of citrate buffer concentrations, in the range of 0.05 M-0.25 M, on the beads disintegration rate. The beads were completely disintegrated within 10 mins in all tested concentrations (0.05 M-0.25 M) of citrate buffer. The citrate buffer is more efficient disintegrating agent than phosphate buffer and it disintegrate the beads in lower concentration.

In addition to the examination of disintegrating agents (which is in the external phase) on the beads disintegration, the influence of various crosslinking ions (Ca⁺², Ba⁺², Fe⁺³, Zn⁺² and Co⁺²) in two different concentrations (which are added in the bead formation process) on the beads disintegration was determined.

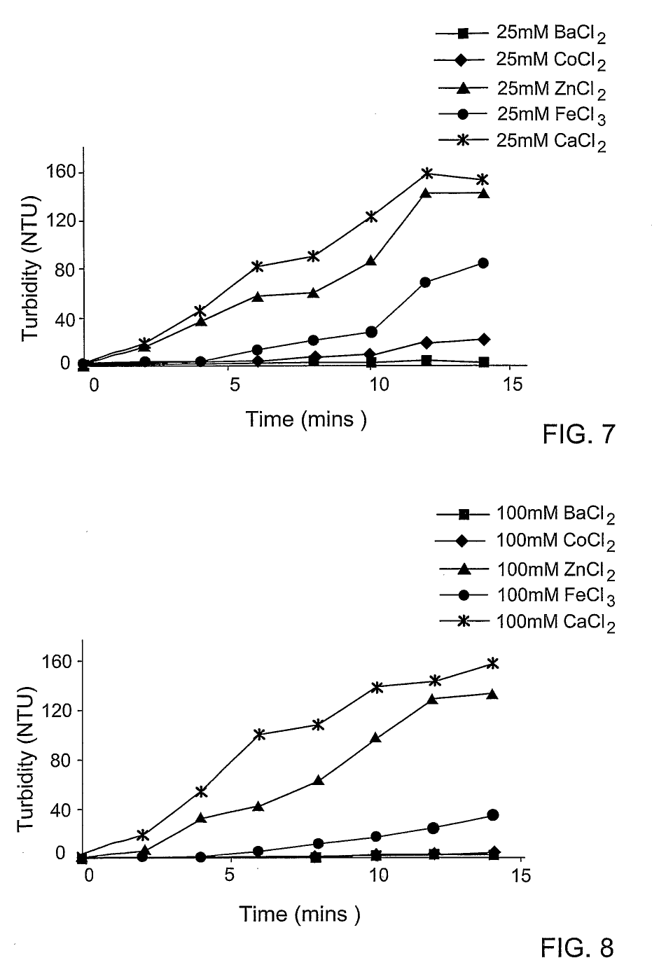

FIGS. 7 and 8 demonstrate the influence of different crosslinking cation on the beads disintegration.

It was found that the beads disintegration depends on the crosslinking ion according to the following order: Ca⁺²>Zn⁺²>Fe⁺³>Co⁺²>Ba⁺². The obtained order is influenced by several parameters such as: the cation valence, the cationic radius, and the ability of the disintegrating agent to competitive on the cation against the alginate polymer.

It was found that by proper selection of disintegrants (type and concentration) and crosslinking (type and concentration) we can control the release rate of the drug.

### EXAMPLE 6

### Microemulsions

Microemulsions were prepared by mixing, without any special equipment—of the solvent (which contains the pre-dissolved drug molecule), the surfactant, co-surfactant and water, at proper composition according to the phase diagram. Than, the obtained microemulsion was mixed with alginate solutions, which upon contact with 2% CaCl₂ solution formed beads in which the microemulsion droplets were dispersed within. The last stage was drying the beads, which lead to formation of drug nanoparticles (size 10-50 nm) dispersed within the bead.

Beads formation: 2.5% Alginate (type LF10/60) solution was mixed with 25% of microemulsion having the composition:

9.1% Brij 96V (polyoxyethylene 10 oleyl ether surfactant)

81.8% Ethanol/Water1:1

9.1% Limonene/Triglymel:1 which contains the dissolved drug.

In an alternative procedure: 2.5% Alginate (type LF 10/60) solution was mixed with 25% microemulsion having the composition:

8% SDS (dodecyl sodium sulfate surfactant)

82% Water

10% BuAc/2-Propanol1:1 containing the dissolved drug.

1.-40. (canceled)

41. A method for producing a drug delivery system comprising nanoparticles or microparticles of a poorly soluble drug dispersed in a polymeric hydrophilic bead and a disintegrate mixed with the bead, comprising:
(i) providing poorly water soluble drug dissolved in organic volatile solvent or mixture of organic volatile solvent with co-solvent that is either miscible or immiscible with water, optionally in the presence of at least one surfactant;
(ii) mixing the drug containing solvent with an aqueous phase comprising at least one surfactant and optionally co-solvent and other emulsification aids at such conditions in which an oil-in-water nanoemulsion or microemulsion is formed;
(iii) mixing the oil-in-water nanoemulsion or microemulsion with water-soluble bead forming polymers to produce a continuous phase of the emulsion which is capable of forming a bead;
(iv) providing conditions enabling bead formation from the continuous phase of (iii) containing nano-microemulsion droplets;
(v) optionally evaporating the volatile organic solvent and the water, thereby obtaining dry beads containing in the polymeric bead dispersed nanoparticles of poorly water soluble drugs.

42. A method according to claim 41, wherein the mixing of the poorly water soluble drug in an organic solvent occurs in the presence of at least one surfactant.

43. A method according to claim 41, wherein the drug containing solvent is mixed within an aqueous phase containing a surfactant, the aqueous phase further containing a co-surfactant and/or co-solvent, and/or electrolytes.

44. A method according to claim 41, wherein the nanoemulsion is prepared by homogenization by a high pressure homogenizer or by a phase inversion method.

45. A method according to claim 41, wherein the microemulsion is formed spontaneously by proper selection of the surfactants, solvent, co-solvent and co-surfactants.

46. A method according to claim 41, wherein at step (iv) the beads are incubated under suitable conditions and for suitable periods of time, with external crosslinking agents.

47. A method according to claim 46, wherein the polymer is an anionic polymer and external crosslinkers are multivalent cations selected from calcium, magnesium, copper, iron, barium and salts of these cations.

48. A method according to claim 46, wherein the polymer is a cation polymer and external crosslinkers are polyvalent anions selected from polyanions or sodium tripolyphosphate.

49. A method for producing a pharmaceutical composition comprising packing the beads obtained in claim 41, within a capsule or tablet.

50. A method according to claim 49, wherein disintegrator is added to the dry beads prior to packing the beads in a capsule or tablet.

51. A method according to claim 50, wherein the disintegrator is selected from chelators and molecules capable of replacing the crosslinking ions.
